(19) United States
(12) Patent Application Publication
(10) Pub. No.: US 2010/0316697 A1
(43) Pub. Date: Dec. 16, 2010

(54) METHODS AND COMPOSITIONS FOR DRUG TARGETING

(75) Inventors: Shmuel Bukshpan, Ramat Hasharon (IL); Gleb Zilberstein, Rehovot (IL)

Correspondence Address: WINSTON & STRAWN LLP;PATENT DEPARTMENT, 1700 K STREET, N.W., WASHINGTON, DC, 20006, US

(73) Assignee: CLEAR DIRECTION LTD., Rehovot (IL)

(21) Appl. No.: 12/599,667

(22) PCT Filed: May 7, 2008
(86) PCT No.: PCT/IL2008/000637

§ 371 Date: Aug. 26, 2010

Related U.S. Application Data
(60) Provisional application No. 60/917,073, filed on May 10, 2007.

(51) Int. Cl. A61K 38/02; C12N 5/071; A61K 9/127; A61K 38/43; A61K 39/395; A61K 31/7088; A61K 31/713; C40B 30/06; A61P 31/04; A61P 31/10; A61P 31/12; A61P 33/00

(52) U.S. Cl. 424/450; 435/375; 514/1.1; 424/94.1; 424/130.1; 514/44 R; 514/44 A; 506/10

(57) ABSTRACT

The present invention provides methods and compositions for targeting a drug to a specific desired location, such as an intracellular location in a mammalian cell, by causing said drug to migrate along a pH gradient to the specific location, where the drug preferentially accumulates at a pH range of the specific location. Accordingly, the invention described herein is based on providing a drug which is “pH matched” with that of a particular location, such that the drug preferentially migrates to and accumulates at the pH or pH range at that location. The location may be a type of tissue, a type of cell, a sub-cellular location or an intracellular location, such as an organelle. Without being bound to any theory, the drug migrates along or across a pH gradient, and stops migrating and accumulates at a location of specific pH or range of pH at which the drug is energetically neutral, or where its diffusion potential is at a minimum.

### FIELD OF THE INVENTION

The present invention relates generally to drug targeting, and more specifically to methods and compositions for intracellular drug targeting and enhanced bioavailabilty, on the basis of drug trapping at a site of specific pH range.

### BACKGROUND OF THE INVENTION

In order to be effective in mammals, a drug must travel a fairly tortuous path from outside the mammal to a specific tissue in which it is to take effect.

Typically, drugs are formulated into medicaments for topical, oral, intravenous or intra-muscular administration. Drugs administered along these routes are often required to be in much higher doses than the actual amount of the drug used in situ. Some drugs are digested in the alimentary canal, and/or are excreted without taking effect. Furthermore, toxic drugs are administered in quantities which may limit their use over time or cumulative use. There is therefore a need to provide improved methods of targeting the transport of the drug to a specific tissue, cell and/or subcellular organelle.

Different strategies may be used to target specific organs and tissues, see for example, “Drug Targeting” Mannhold et al, Methods and Principles in Medicinal Chemistry, Wiley, published online 11 Oct. 2001, which is incorporated herein in its entirety.

There are two major kinds of targeted drug delivery. The first one is active targeted drug delivery, such as antibody drugs, wherein the antibody has high specificity for a certain antigen. The second one is passive targeted drug delivery employing for example, an enhanced permeability and retention (EPR) effect. This EPR is a property by which certain sizes of molecules, typically liposomes or macromolecular drugs, tend to accumulate in tumor tissue much more than they do in normal tissues. The general explanation that is given for this phenomenon is that, in order for tumor cells to grow quickly, they must stimulate the production of blood vessels (VEGF) and thus have effective uptake routes for various molecules.

Sinha and Rachna Kumria disclose a prodrug approach to colonic drug delivery (in Pharmaceutical Research 18(5) May 2001, 557-564). One of the approaches used for colon specific drug delivery is the formation of a prodrug which optimizes drug delivery and improves drug efficacy. Many prodrugs have been evaluated for colon drug delivery. These prodrugs are designed to pass intact and unabsorbed from the upper gastrointenstinal tract and undergo biotransformation in the colon releasing the active drug molecule. This biotransformation is carried out by a variety of enzymes, mainly of bacterial origin present in the colon (e.g. azoreductase, glucuronidase, glycosidase, dextranase, esterase, nitroreductase, cyclodextranase, etc.).

U.S. Pat. No. 7,135,547 to Gengrinovitch discloses peptide conjugated anti-cancer prodrugs. This patent relates to pharmaceutical compositions that include a targeting peptide, a protease specific cleavable peptide, and a chemotherapeutic drug that when conjugated are substantially inactive, but upon degradation of the cleavable sequence by a proteolytic enzyme abundant in or within the target cancer cell, the chemotherapeutic drug is released and becomes active, and to methods of use of these compositions for treatment of cancer.

U.S. Pat. No. 7,208,314 to Monahan et al describes a system relating to the delivery of desired compounds (e.g., drugs and nucleic acids) into cells using pH-sensitive delivery systems. The system provides compositions and methods for the delivery and release of a compound to a cell.

U.S. Pat. No. 5,851,789 to Simon et al discloses administering to a subject an agent capable of modifying intracellular pH, either alone or in combination with an anti-cancer drug, to counteract multidrug resistance.

U.S. Pat. No. 7,108,863 to Zalipsky et al discloses a method for increasing accumulation of a therapeutic agent in cellular nuclei which comprises providing and administering liposomes comprising a pH-sensitive lipid; a lipid derivatized with a hydrophilic polymer; a targeting ligand e.g. an antibody, and the therapeutic agent entrapped therein. According to the disclosure, the accumulation of the agent in the nucleus of the target cell is at least two-fold higher when compared to intracellular concentration of the agent delivered by similar liposomes lacking the releasable bond and/or the targeting ligand.

Cellular transmembrane pH gradient dependent cytotoxicity has been observed in specific weak acid chemotherapeutics (S. V. Kozin et al. Cancer Research 61, 4740, Jun. 15, 2001). It has further been disclosed that tamoxifen, like monensin and bafilomycin A1, causes redistribution of weak base chemotherapeutics, such as adriamycin from the acidic organelles to the nucleus in drug-resistant cells (Altan et al Proc Natl Acad Sci USA 96, 4432-4437, 1999).

A mechanism of selective transport and localization of proteins within living cells based on pH-induced protein trapping has been disclosed by some of the inventors of the present invention, on the basis of observations in artificial systems with fixed non-uniform pH distribution and in living cells (Baskin et al. Physiol Biol 3,101-106, 2006).

Not only does a drug delivery route need to be mapped carefully to find an optimal delivery route of the drug to the specific tissue, but it needs to be ascertained that the drug is taken up by the tissue and is active therein.

There is still a need to develop drugs and methods for highly selective targeting thereof within a mammalian body to maximize the effectiveness thereof.

The prior art does not disclose or teach a method of targeting a drug to an intracellular location wherein the method comprises causing the drug to migrate along an intracellular pH gradient to the intracellular location, wherein the drug preferentially accumulates at a pH range of said intracellular location.

### SUMMARY OF THE INVENTION

It is an object of some aspects of the present invention to provide methods and compositions for improved drug targeting on the basis of drug trapping at a site of specific pH.

It is a further object of some aspects of the present invention to provide methods and compositions for improved drug targeting to a cell on the basis of drug trapping at a site of specific pH.

In some embodiments of the present invention, improved methods and compositions are provided for drug delivery within a cell on the basis of drug trapping at an intracellular site of specific pH.

The inventors of the present invention have surprisingly observed that a protein may preferentially distribute in a subcellular region of a living cell at a certain localized pH range. Accordingly, a protein or peptide may be transported in tissue and within a cell across or along a pH gradient. Without being bound to any theory, the mechanism may be based on pH-dependent protein trapping. This intrinsic property of proteins may be exploited for the targeted delivery of a drug to specific intracellular locations where the drug activity is needed for treatment of a specific disease or disorder.

Accordingly, the invention described herein is based on providing a drug which is “pH matched” with that of a particular location, such that the drug preferentially migrates to and accumulates at the pH or pH range at that location. The location may be a type of tissue, a type of cell, a sub-cellular location or an intracellular location, such as an organelle. Without being bound to any theory, the drug migrates along or across a pH gradient, and stops migrating and accumulates at a location of specific pH or range of pH at which the drug is energetically neutral, or where its diffusion potential is at a minimum.

There is thus provided according to a first aspect of the present invention, a method for targeting a drug to an intracellular location in a eucaryotic cell where the drug takes effect, comprising;
causing the drug to migrate along a pH gradient to the intracellular location, whereby the drug preferentially accumulates at a pH range in the intracellular location.

In one embodiment, the eucaryotic cell is a mammalian cell. In some cases, the mammalian cell is selected from a brain cell, a skin cell, a lung cell, a nerve cell, a heart cell, an alimentary canal cell, a cancer cell, a blood cell, a urinary tract cell and an infected cell. According to some embodiments, the cancer cell is selected from a tumor cell, a leukemia cell, a carcinoma cell, a lymphoma cell, a sarcoma cell, a metastatic cell, and a multidrug resistant cancer cell. The infected cell may be selected from, a parasite-infected cell, a virus-infected cell and a prion-infected cell.

Sometimes, the mammalian cell is part of a tissue. In some cases, the tissue has a disease or disorder. The disease or disorder may be an infection selected from a bacterial infection, a fungal infection, a viral infection, a prion infection and a parasitical infection.

According to some embodiments, the method further comprises delivering the drug into a specific tissue or cell type. In some embodiments, the method comprises delivering the cell intracellularly to a target cell. In some embodiments, the method comprises delivering the drug into a specific target tissue.

According to some embodiments, the delivering step comprises providing the drug in a formulation comprising at least one drug delivery component. The at least one drug delivery component may comprise at least one molecule or moiety which shifts the trapping probability of the drug in an intracellular pH gradient. Examples of a drug delivery component include without limitation, a liposome, a nucleic acid vector, a sialyl Lewis receptor, folate EGF, an anti-target antibody, a pH-sensitive delivery system, a pH controlled drug release system, a time-controlled drug release system, a pressure-controlled drug release system, a molecular positive charging system, a receptor binding component, a chimeric peptide, a cathepsin-sensitive component; a buffering system, an encapsulation system, a blood-brain barrier traversing component, a component susceptible to phagocytosis, a component susceptible to pinocytosis, a component susceptible to transcytosis and a component susceptible to endocytosis.

The anti-target antibody may be selected from an anti-B-FN antibody, an anti-CD20 antibody, and an anti-IL-2Rα antibody.

According to some embodiments of the present invention, the drug comprises at least one of the following: a peptide, a protein, an enzyme, an antibody, an anti-inflammatory drug, an anti-cancer drug, an antibiotic, a drug delivery component, a sense nucleic acid, an anti-sense nucleic acid, a covalently bound adjunct, a receptor binding component, a prodrug, a cleavable sequence, and an active fragment of any of the above.

According to some embodiments, causing the drug to migrate along a pH gradient comprises providing the drug together with at least one moiety which shifts the trapping probability of the drug along the intracellular pH gradient. According to some embodiments, the at least one moiety which shifts the trapping probability of the drug along the intracellular pH gradient is selected from the group consisting of a peptide, a protein and a protein fragment. According to some embodiments, providing the drug together with the moiety which shifts the trapping probability of the drug along the intracellular pH gradient comprises preparing a covalent conjugate of the drug and said moiety. According to some embodiments, preparing a covalent conjugate comprises use of a cross-linking reagent. According to some embodiments, preparing a covalent conjugate comprises chemical conjugation. According to some embodiments, preparing a covalent conjugate comprises recombinant expression of a fusion protein.

According to some embodiments, the drug comprises at least one moiety which shifts the trapping probability of the drug along the intracellular pH gradient. According to some embodiments, the drug and said moiety are present together in a covalent conjugate. In one embodiment, the covalent conjugate is a fusion protein.

In the method described herein, the pH range may be less than 3 pH points, less than two pH points or even less than one pH point.

According to some embodiments, the intracellular location is selected from a location in a nucleus, in an organelle, in cytoplasm and in cytosol. The organelle may be selected from the group consisting of a mitochondrion, a ribosome, a Golgi apparatus, an endoplasmic reticulum and a centrasome.

According to some further embodiments, the drug may migrate to the intracellular location within a specified period of time, for example in less than 5 minutes or in less than two minutes.

The drug may be activated, according to some embodiments, in the vicinity of at least ATP or a phosphate group.

The drug may cease to migrate at an energetically favorable intracellular location on the basis of the pH or pH range at that location.

In one embodiment, the method further comprises modifying the pH gradient in the cell by the addition of a pH modifying agent. In one embodiment, the pH modifying agent is selected from the group consisting of monensin, bafilomycin A₁ and tamoxifen. In various embodiments, the pH modifying agent is administered prior to, concurrent with or following administration of the drug.

There is thus provided according to another aspect of the present invention, a composition for targeting a drug to an intracellular location in a eucaryotic cell where the drug takes effect, comprising;
a drug adapted to migrate along a pH gradient to the intracellular location by having an enhanced trapping probability matched to a pH range of the intracellular location, whereby the drug is active or activated in the intracellular location; and
an aqueous carrier.

According to some embodiments, the drug further comprises at least one molecule or moiety which shifts the trapping probability of the drug along the intracellular pH gradient.

According to some embodiments, the at least one moiety which shifts the trapping probability of the drug along the intracellular pH gradient is selected from the group consisting of a peptide, a protein and a protein fragment. According to some embodiments, the drug comprises at least one moiety which shifts the trapping probability of the drug along the intracellular pH gradient. According to some embodiments, the drug and said moiety which shifts the trapping probability of the drug along the intracellular pH gradient are present together in a covalent conjugate. In one embodiment, the covalent conjugate is a fusion protein.

The composition may further comprise at least one drug delivery component for delivering said drug from outside the body of a mammal to said cell or for delivering said drug from an extracellular location to the intracellular region of the target cell.

In some cases, the at least one drug delivery component comprises at least one molecule or moiety which shifts the trapping probability of the drug in a pH gradient. Examples of a drug delivery component include without limitation, a liposome, a nucleic acid vector, a sialyl Lewis receptor, folate EGF, an anti-target antibody, a pH-sensitive delivery system, a pH controlled drug release system, a time-controlled drug release system, a pressure-controlled drug release system, a receptor binding component, a chimeric peptide, a cathepsin-sensitive component; a buffering system, an encapsulation system, a blood-brain barrier traversing component, a component susceptible to phagocytosis, a component susceptible to pinocytosis, a component susceptible to transcytosis and a component susceptible to endocytosis.

The anti-target antibody may be selected from an anti-B-FN antibody, an anti-CD20 antibody, and an anti-IL-2Rα antibody.

According to some embodiments, the drug may be selected from a peptide, a protein, an enzyme, an antibody, an anti-inflammatory drug, an anti-cancer drug, an antibiotic, a drug delivery component, a sense nucleic acid, an anti-sense nucleic acid, a covalently bound adjunct, a receptor binding component, a prodrug, a cleavable sequence, an active fragment thereof and combinations thereof.

Some further embodiments of the present invention are directed to a method of drug screening for a drug active at a sub-cellular location in a mammalian cell, comprising:
mapping an intracellular pH distribution of a cell so as to define a pH range of a sub-cellular location; and
screening drugs from a drug library to find one or more drugs having an increased probability of accumulating at the pH range of the sub-cellular location.

This method may further include testing the one or more drugs to verify an activity thereof in the sub-cellular location.

There is thus provided according to some embodiments of the present invention, a method of drug design for a drug active at a sub-cellular location in a mammalian cell, comprising:
mapping an intracellular pH distribution of the cell so as to define a target pH range of a sub-cellular target location; and
evaluating and sorting drugs in a drug library according to their increased probability for accumulating at a specific pH range to form target pH drug groups;
matching the target pH drug groups to the target pH range to select one or more matched drug groups; and
designing a delivery system for at least one drug from the one or more matched drug groups suitable for delivery for the at least one drug to the sub-cellular location so as to provide at least one drug active at the sub-cellular location in the mammalian cell.

In one embodiment, the cell is one which exhibits multidrug resistance (MDR). In some cases the cell exhibiting MDR is a cancer cell.

Some embodiments of the present invention are directed to a method for identifying a defective protein comprising:
mapping an intracellular pH distribution of a standard wild type active form of the protein in a standard reference mammalian cell to form a standard reference pH distribution map of the protein;
mapping an intracellular pH distribution of an isolated form of the protein in the standard reference mammalian cell to form a pH distribution map of the isolated protein; and
comparing the pH distribution map of the isolated protein with the standard reference pH distribution map to determine if the isolated protein is defective. The present invention will be more fully understood from the following detailed description of various embodiments thereof, the drawings, and Examples.

### BRIEF DESCRIPTION OF THE DRAWINGS

FIG. 1 is a simplified flow chart of a method for targeted drug design, in accordance with a preferred embodiment of the present invention;

FIG. 2 is a simplified flow chart of a method for targeted drug design and selection, based upon an intracellular target pH, in accordance with a preferred embodiment of the present invention;

FIG. 3 is a simplified flow chart of a method for targeted prodrug design, based upon an intracellular target pH, in accordance with a preferred embodiment of the present invention.

FIG. 4 is a simplified flow chart of a method for rational design and optimized selection of a drug based upon an intracellular target pH, in accordance with a preferred embodiment of the present invention;

FIG. 5 shows the effect of linking the heterologous proteins lactoglobulin and Concanavalin A to shift the trapping probability in a pH gradient. Lactoglobulin (panel A), Concanavalin A (panel B) and a crosslinked conjugate of lactoglobulin and Concanavalin A (panel C) were assessed for their distribution and preferential accumulation in an immobilized pH gradient.

FIG. 6 shows the effect of linking the heterologous proteins lactoglobulin and bovine serum albumin to shift the trapping probability in a pH gradient. Lactoglobulin (panel A), bovine serum albumin (panel B) and a crosslinked conjugate of lactoglobulin and bovine serum albumin (panel C) were assessed for their distribution and preferential accumulation in an immobilized pH gradient.

### DETAILED DESCRIPTION

This invention is directed to methods and compositions for targeted drug delivery based on the migration of the drug along an intracellular pH gradient.

A protein, for example, may be transported within a cell across or along a pH gradient, as has been shown previously (Baskin et al. Physiol Biol 3,101-106, 2006, incorporated herein its entirety by reference). The protein may migrate and settle in a subcellular region of the cell, such as an organelle, in which the localized pH range may be energetically favorable for the protein, relative to the other regions in the cell, through which the protein migrated. This subcellular region may in some cases, have a pH around or equal to the pH at which the protein has an increased probability of accumulation. Without being bound to any theory, the mechanism of protein migration may be based on pH-induced protein trapping.

In order to cause the drug to accumulate at a certain intracellular location, the drug may be provided with at least one molecule or moiety which shifts the trapping probability of the drug along the intracellular pH gradient.

The molecule or moiety which shifts the pH trapping property of the drug may be covalently or non-covalently bound to the drug itself. Alternately or in addition, the molecule or moiety which shifts the pH trapping property of the drug may be a separate component of the drug composition or formulation, such as in a drug delivery component.

In some other cases, there may be several such molecules or moieties which shift the pH trapping property of the drug, some of which are attached to the drug (covalently, non-covalently or a combination thereof), and some being in the composition or formulation thereof.

“Covalent association”, “covalent bond” and associated grammatical forms, such as “covalently associated” and “covalently bound” respectively, refer interchangeably to an intermolecular association or bond which involves the sharing of electrons in the bonding orbitals of two atoms. “Non-covalent association”, “non-covalent bond” and associated grammatical forms refer interchangeably to intermolecular interaction among two or more separate molecules or molecular entities which does not involve a covalent bond. Intermolecular interaction is dependent upon a variety of factors, including, for example, the polarity of the involved molecules, and the charge (positive or negative), if any, of the involved molecules. Non-covalent associations are selected from ionic interactions, dipole-dipole interactions, van der Waal's forces, and combinations thereof.

A number of reagents capable of cross-linking molecules such as peptides are known in the art, including for example, azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-.gamma.-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, formaldehyde and succinimidyl 4-[N-maleimidomethyl]cyclohexane-1-carboxylate.

By “isoelectric point” of a molecule is meant the pH of an aqueous solution in which a molecule, such as a zwitterionic protein, is contained wherein the molecule has no net electrical charge.

By “pH matching” is meant that a pH range of a drug, such as a protein, at which it preferentially accumulates (PA), after migrating along a pH gradient, is matched with a pH range of a sub-cellular/intracellular location (SCP), such as an organelle. Without being bound to any theory, the drug may stop migrating at a location of specific pH or range of pH, wherein at that location the drug is energetically neutral 1, or its diffusion potential is at a minimum.

It can be understood from Baskin et al., that the mechanism of pH-induced molecule migration need not be limited to proteins, but may be applied to a large number of biological molecules and drugs.

These biological molecules or drugs may be selected from, but are not limited to comprise, at least one of the following; an amino acid, a peptide, a protein, an enzyme, an antibody, an anti-inflammatory drug, an anti-cancer drug, an antibiotic, a drug delivery component, a sense nucleic acid, an anti-sense nucleic acid, a covalently bound adjunct, a receptor binding component, a prodrug, a cleavable sequence, and an active fragment of any of the above.

According to some embodiments, the drug or a portion thereof exhibits amphoteric/zwitterionic activity, such that in an aqueous solution, it is electrically neutral at a certain pH.

In order to transport the drug from the point of delivery to the mammal to the cell or tissue in which it is to take effect, the drug may be provided in a composition or formulation comprising at least one drug delivery component. According to some other embodiments, the drug may not be formulated.

In some cases, the at least one drug delivery component comprises at least one molecule, such as a peptide, which shifts the trapping probability of the drug in an intracellular pH gradient. Additional examples of a drug delivery component include, without limitation, a liposome, a nucleic acid vector, a sialyl Lewis receptor, folate EGF, an anti-target antibody, a pH-sensitive delivery system, a pH controlled drug release system, a time-controlled drug release system, a pressure-controlled drug release system, a receptor binding component, a chimeric peptide, a cathepsin-sensitive component; a buffering system, an encapsulation system, a blood-brain barrier traversing component, a component susceptible to phagocytosis, a component susceptible to pinocytosis, a component susceptible to transcytosis and a component susceptible to endocytosis.

The molecule which shifts the trapping probability of the drug in a pH gradient may be, according to some embodiments, a peptide, a protein or a protein fragment.

The drug and the moiety which shifts the trapping probability of the drug along the intracellular pH gradient may be provided as a fusion protein prepared using recombinant DNA methodology and expression in a suitable host cell, as is known in the art (see for example Maniatis et al., Molecular Cloning: A Laboratory Manual, Cold Spring Harbor Laboratory, 1988). Accordingly, an expression vector or plasmid comprising DNA segments which direct the synthesis of the fusion protein may be expressed in a variety of host cells, including E. coli, other bacterial hosts, yeasts and various higher eucaryotic cells such as COS, CHO and HeLa cell lines.

The recombinant DNA sequence encoding the fusion protein will be operably linked to appropriate expression control sequences for each host. For E. coli this includes a promoter such as the T7, trp, or lambda promoters, a ribosome binding site and preferably a transcription termination signal. For eucaryotic cells, the control sequences will include a promoter and preferably an enhancer derived from immunoglobulin genes, SV40, cytomegalovirus, etc., and a polyadenylation sequence, and may include splice donor and acceptor sequences. The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the ampicillin resistance gene.

Once expressed, the recombinant fusion proteins can be purified according to standard procedures of the art, including ammonium sulfate precipitation, affinity columns, column chromatography, gel electrophoresis and the like (see, generally, R. Scopes, “Protein Purification”, Springer-Verlag, N.Y. (1982)). Substantially pure compositions of at least about 90 to 95% homogeneity are preferred, and 98 to 99% or more homogeneity most preferred, for pharmaceutical uses. According to some other embodiments, an extracellular pH may be altered to enhance or improve the intracellular uptake and/or distribution of the drug, as disclosed for example by Gerweck et al. Mol. Cancer. Ther. 2006 5(5):1275-1279, incorporated herein by reference in its entirety, relating to changing a ratio of chloroamucil- to doxorubicin-uptake into tumor cells. The method of changing an extracellular pH in order to alter the intracellular distribution of a drug is also reported in Keizer et al. (Cancer Research 49:2988-2993 (1989) incorporated herein by reference in its entirety). Keizer reported that using a different external pH value during drug exposure, it was possible to show that there is a gradual change in subcellular drug distribution, that is correlated with the level of doxorubicin resistance.

In order for a drug for intravenous injection to be effective in a brain cell, for example, it must be introduced into a mammalian body, pass along a route, such as via the blood stream, traverse the blood brain barrier, travel to the relevant part of the brain and enter the cells at that part of the brain.

In order for an oral drug to be effective inside a tumor in the liver, it must be introduced into a mammalian body, pass along a route, such as via the alimentary canal, avoid digestion or excretion, pass from the alimentary canal via a second route, such as via the bloodstream, to the liver and “find the tumor” and then enter that tumor.

Thus, very different strategies may be used to deliver an oral and an intravenous drug to a target within the body. An excellent review of these strategies, together with practical examples, is provided in “Drug Targeting” Mannhold et al., Methods and Principles in Medicinal Chemistry, Wiley, published online 11 Oct. 2001, which is incorporated herein in its entirety. However, upon review of the drug targeting methods disclosed, there is insufficient information provided on how to perform intracellular drug targeting.

The intracellular targeting methods of the present invention may, according to some embodiments, be combined with the delivery methods of the prior art to provide optimized drug targeting methods and compositions.

In some embodiments, the method of the invention may further comprise modifying the pH gradient in a desired target cell or tissue type by the addition of a pH modifying agent. It is known that in some disease conditions and/or in response to certain drugs (for example multidrug resistance exhibited by certain cancer cells following treatment with anti-neoplastic agents), the “native” pH gradient of a cellular or intracellular location is disturbed. To counteract such a disturbance, and to restore the pH gradient, a pH modifying agent may be administered. Suitable pH modifying agents include without limitation, monensin, bafilomycin A₁ and tamoxifen. The pH modifying agent may be administered prior to, concurrent with or following administration of the drug. Accordingly, the efficacy of drug targeting according to the invention may be enhanced, due to the restoration or creation of a favorable pH gradient in the target tissue or cellular location where drug activity is sought.

The drugs and drug formulations of this invention are particularly useful for parenteral administration, i.e., subcutaneously, intramuscularly or intravenously. The compositions for parenteral administration will commonly comprise a solution of the antibody or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of undesirable matter. These compositions may be sterilized by conventional, well known techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate and the like. The concentration of drug in these formulations can vary-widely, and will be selected primarily based on extablished properties of the drug, fluid volumes, viscosities, body weight and the like in accordance with the particular mode of administration selected.

Actual methods for preparing parenterally administrable compositions will be known or apparent to those skilled in the art and are described in more detail in such publications as Remington's Pharmaceutical Science, 15th ed., Mack Publishing Company, Easton, Pa. (1980), which is incorporated herein by reference.

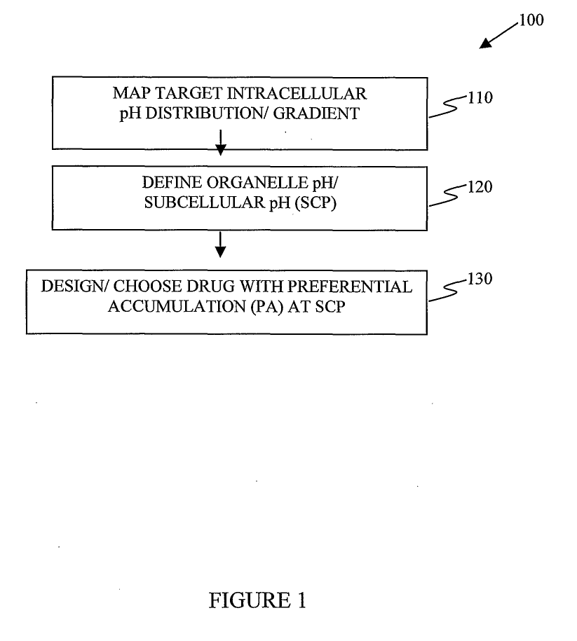

Reference is now made to FIG. 1, which is a simplified flowchart 100 of a method for targeted drug design, in accordance with an embodiment of the present invention.

In a mapping step 110, a pH distribution map or pH gradient map of a target cell is made, using inter alia, the methods described in Baskin et al. ibid. Typically, this step will define sub-cellular locations and regions having a small/limited pH range. The pH of intracellular organelles is also defined during this process. A pH map may be devised showing the pH of the cell in a two-dimensional or three dimensional image. The pH map may be superimposed on another map/image of the cell showing the various organelles and subcellular regions. Various alternative techniques known in the art may be used to compose a two-dimensional and/or three-dimensional pH map.

In some cases, the pH mapping in this step may be provided as raw data, such as hydrogen ion concentration, which requires conversion/mathematical manipulation, in order to define the pH.

According to some embodiments, an additional defining step 120 for defining the pH may be required, such as to extract the image data and to define all regions having a certain pH. Additionally or alternatively, pH gradients may be mapped. At the end of these two steps (110-120), a full definition of the pH of the subcellular locations and of the various organelles will be known and mapped. This may require the use of image analysis techniques, known in the art For example the pH map may be superimposed onto another image of the organelles in the cell. It may be found, for example, that a first organelle, to which a drug is to be delivered, has a pH range of 5.5-6, whereas a second organelle has a pH range of 7-7.5. In another example, a certain cytosolic location has a pH of 7.

Additionally, an energetic analysis of the drug along a pH gradient may be performed in vitro or in vivo to map the diffusion potential of the drug along a pH gradient, at one or more different temperatures, and to determine the pH or pH range at which the drug ceases to migrate (see Baskin et al., ibid).

In a drug design step, 130, a drug will be designed for application to the first organelle mentioned hereinabove. This step may include many sub-steps. It should be understood that a drug to be effected in the first organelle will be designed differently to a drug for the second organelle. The drug design may include any of the following sub-steps:
The pH to which the drug preferentially migrates (PA) of a first potential drug will be defined.
The isoelectric point of the potential drug will be defined.
Its migration along a pH gradient may be mapped in vitro in a system simulating the in vivo conditions (see Baskin et al ibid.).
The drug's migration under various electric potential fields may be mapped.
The drug's migration under various thermal gradients may be mapped.
Analysis of the quantity and quality of the drug activity in vitro/in vivo may be performed.
The drug may be provided with at least one molecule which shifts the trapping probability of the drug along an intracellular pH gradient.

According to some embodiments, after evaluating and designing the drug, the preferential pH of the designed drug for accumulation (PA) will be determined If the PA is similar or equal to the pH range of the organelle, then the process for targeted drug design may be complete.

In some cases, the drug will be active at that pH. In other cases, there may be a need to make the drug more bioavailable and/or to activate the drug. The former may be performed, for example, by modifying the drug with additional positive charge, as is known in the art. The latter, may be performed by providing a localized increase in ATP or phosphate ions.

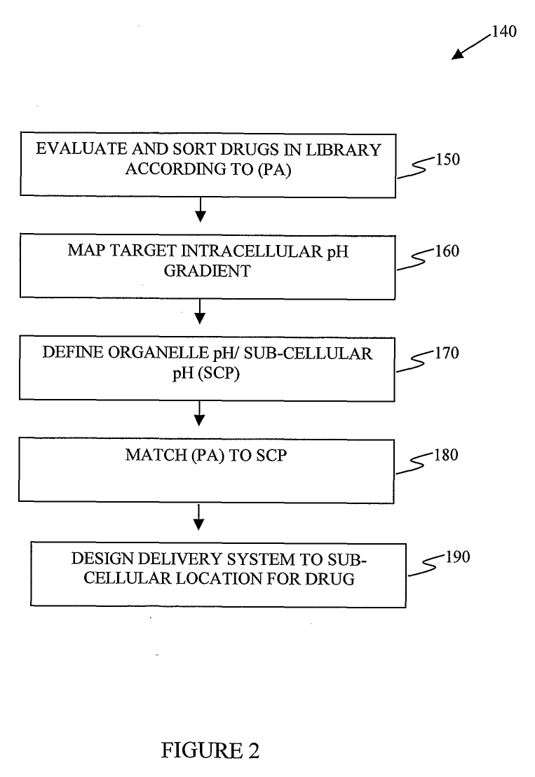

Turning to FIG. 2, a simplified flowchart 140 can be seen of a method for targeted drug design and selection, based upon an intracellular target pH, in accordance with some embodiments of the present invention.

There are many online and offline databases, search engines and sources of information (named collectively herein “libraries”) comprising data relating to drugs such as proteins. These libraries may be mapped to select a group of drugs having a certain activity, such as a catalase activity. All drugs of the group may be evaluated and sorted according to their PA. For example, there may be commercial sources of catalase, from one or more bacterial or fungal sources, genetically modified enzymes available from a national depositary, commercial mammalian sources of the enzyme, heat resistant engineered molecules of catalase, catalase with low coenzyme requirement. Each of these enzymes may have a different PA (pH to which they preferentially migrate), which may have been determined previously, and this data may be available in the library or libraries.

Thus, in step 150, the PA values of some or all of the above catalase molecules available from the libraries may be evaluated and sorted to determine the different catalase molecules having a PA which falls within a certain pH range. For a certain drug or drug type, the PA thereof may thus be mapped from the libraries.

This step (150) may be performed for many types of drugs, and is not limited to proteins or enzymes.

The catalase may be required for a certain adrenal cortex cell, in which the peroxisomes have a non-functional catalase, or catalase in too low a copy number.

In a mapping step 160, a pH distribution map or pH gradient map of a target cell, such as the adrenal cortex cell is made, using, inter alia, the methods described in Baskin et al. ibid. Typically, this step will define sub-cellular locations and regions having a small/limited pH range.

The pH of intracellular organelles is also defined during this process. A pH map may be devised showing the pH of the cell in a two-dimensional or three dimensional image. The pH map may be superimposed on another map/image of the cell showing the various organelles and subcellular regions. Various alternative techniques known in the art may be used to compose a two-dimensional and/or three-dimensional pH map. Any additional mapping or defining as described hereinabove with respect to step 120 (FIG. 1), may be performed too.

In some cases, the pH mapping in this step may be provided as raw data, such as hydrogen ion concentration, which requires conversion/mathematical manipulation, in order to define the pH.

In a defining step 170, the organelle or sub-cellular region pH (SCP) may be defined. Thus, for example, the pH of the peroxisomes of the adrenal cortex cells may be defined.

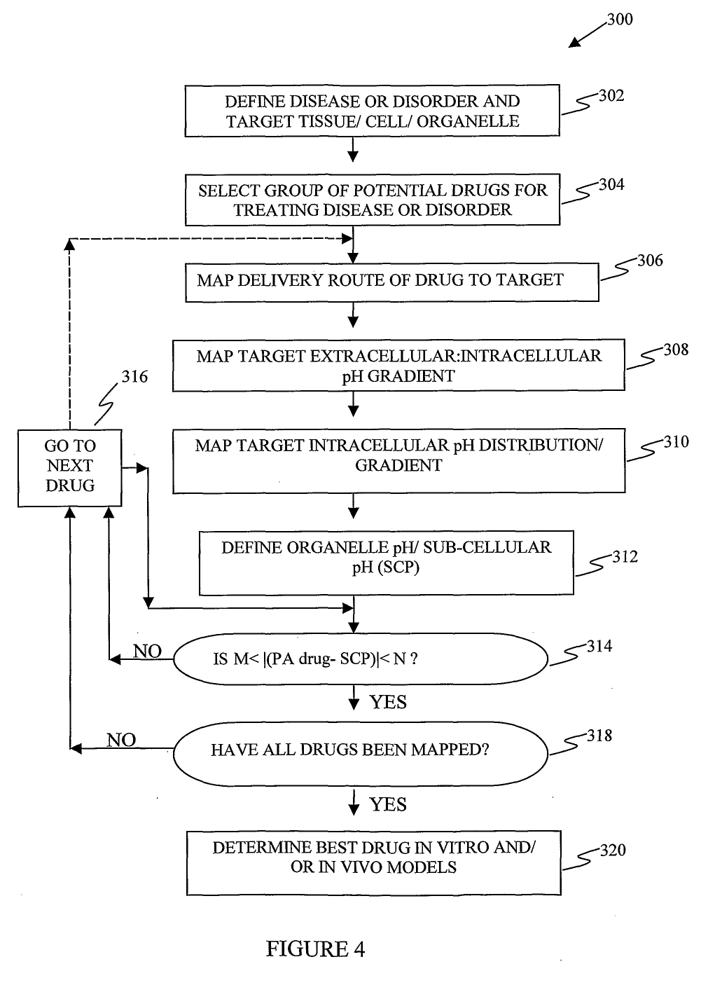

In a matching step 180, the PA values of the catalase molecules may be matched with the SCP of the peroxisome (pH matching as defined hereinabove). It should be understood that this may be an iterative process, involving several sub-steps. In some cases, this step may be performed at least partially by using a computer program. The data may be stored in one or more memories and retrieved therefrom for performing this step. Additionally, the results may also be stored in the memory (see further discussion with respect to FIG. 4 hereinbelow).

Of all the various catalase molecules mapped from the library in step 150, one or more of them may have a similar or same PA and matched to the SCP in step 180.

In step 190, a suitable targeted delivery system may be designed for the molecules chosen in step 180.

One or more of the following sub-steps may be performed to the chosen molecules.
A drug delivery component, such as those listed hereinabove, may be added to the drug to match the requirements of the target pH range. In particular, a pH-sensitive delivery system, such as, but not limited to that described in U.S. Pat. No. 7,208,314, may be used.
The drug may be covalently bonded to another molecule to improve the migration abilities of the drug from the point of entry to the cell to the organelle or sub-cellular location.
The drug may be provided with at least one molecule which shifts the trapping probability of the drug along the intracellular pH gradient.
The drug may be modified by providing it with a positive charge.
Some or all of the above steps (150-180) may be performed on the drug after formulation/and/or after addition of one or more drug delivery components and/or after covalent modification thereof and/or after genetic engineering thereof so as to determine the energetic and pH characteristics (such as PA and/or isoelectric point) following these manipulations.

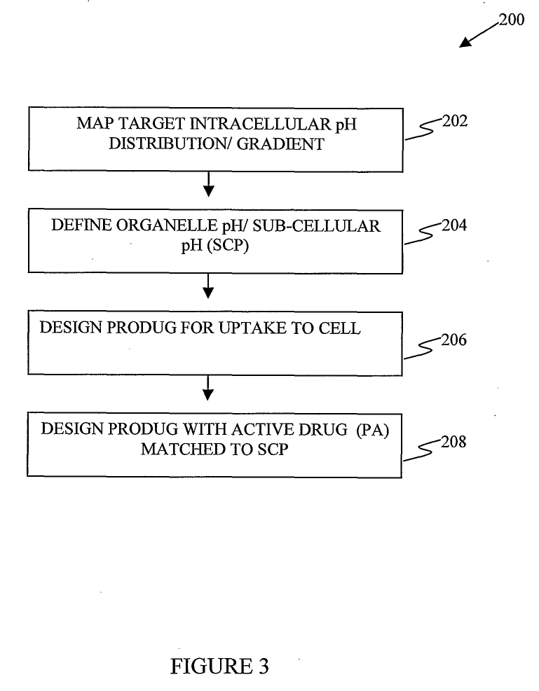

FIG. 3 is a simplified flowchart 200 of a method for targeted prodrug design, based upon an intracellular target pH, in accordance with an embodiment of the present invention.

In a mapping step 202, a pH distribution map or pH gradient map of a target cell is made, using, inter alia, the methods described in Baskin et al. ibid. Typically, this step will define sub-cellular locations and regions having a small/limited pH range. This step may be similar or identical to step 110 (FIG. 1). The pH of intracellular organelles is also defined during this process. A pH map may be devised showing the pH of the cell in a two-dimensional or three dimensional image. The pH map may be superimposed on another map/image of the cell showing the various organelles and subcellular regions. Various alternative techniques known in the art may be used to compose a two-dimensional and/or three-dimensional pH map.

In some cases, the pH mapping in this step may be provided as raw data, such as hydrogen ion concentration, which requires conversion/mathematical manipulation, in order to define the pH.

According to some embodiments, an additional defining step 204 for defining the pH may be required, such as to extract the image data and to define all regions having a certain pH. Additionally or alternatively, pH gradients may be mapped. At the end of these two steps (202-204), a full definition of the pH of the subcellular locations and of the various organelles will be known and mapped. This may require the use of image analysis techniques, known in the art. For example the pH map may be superimposed onto another image of the organelles in the cell. It may be found, for example, that a first organelle, to which a drug is to be delivered, has a pH range of 5.5-6, whereas a second organelle has a pH range of 7-7.5. In another example, a certain cytosolic location has a pH of 7.

Additionally, an energetic analysis of the drug along a pH gradient may be performed in vitro/in vivo to map the diffusion potential of the drug along a pH gradient, at one or more different temperatures, and to determine the pH at which the drug ceases to migrate (see Baskin et al., ibid).

The extracellular pH may be defined too. Thereafter, the energetic and/or pH requirements for the drug to enter the cell may be defined. It may then be understood that, in order for the drug to be effective at the specific target intracellular location and for the drug to easily be transferred into the cell, one or more targeted drug delivery systems may be required. Additionally, in order to be conveyed into the cell, the drug may need to be in a prodrug form so as to retain its activity for use in the cell.

In a prodrug design step, 206, the prodrug drug will be designed for transfer into the cell and for delivery to the intracellular target.

This step may include many sub-steps. The prodrug design may include any of the following sub-steps:
The PA of a potential drug and/or prodrug will be defined. The prodrug may be designed to activate the drug at a certain intracellular pH by methods known in the art (see for example, U.S. Pat. No. 6,030,997, incorporated herein by reference in its entirety).
The isoelectric point of a potential drug and/or prodrug will be defined.
The migration of the drug and/or prodrug along a pH gradient may be mapped in vitro in a system simulating the in vivo conditions (see Baskin et al ibid.).
The migration of the drug and/or prodrug under various electric fields may be mapped.
The migration of the drug and/or prodrug under various thermal gradients may be mapped.
The conformation of the drug, such as a protein, may be altered by chemical treatment so as to change its isoelectric point and/or its charge.
The prodrug/drug may be provided with at least one molecule which shifts the trapping probability of the drug along the intracellular pH gradient.
The drug may be genetically engineered to provide a different conformation.
Analysis of the quantity and quality of the drug and/or prodrug activity in vitro/in vivo may be performed.
The drug may be covalently bonded to one or more other molecules to improve the migration abilities of the drug from the point of entry to the cell to the organelle or sub-cellular location.
The drug may be covalently bonded to one or more other molecules to prevent the drug being active at locations distant from the target cell, but to allow the drug to be active proximal to and/or within the target cell (see WO02/20715 to Gengrinovitch, which is incorporated herein by reference).

In a next designing step 208, after evaluating and designing the drug and prodrug, the designed drug's and or prodrug's PA will be determined. In some cases, if the PA is similar or equal to that pH range of the organelle, then the process for targeted drug design will be complete.

FIG. 4 is a simplified flowchart 300 of a method for rational design and optimized selection of a drug based upon an intracellular target pH, in accordance with an embodiment of the present invention.

In a first defining step 302, the disease or disorder that the mammal suffers from is defined, and the target tissue/cell/organelle is located. For example, the human or other mammalian patient may exhibit some symptoms, which may be analyzed by one or more professionals selected from a researcher, a medical practitioner, a laboratory technician and a paramedic. The practitioner may request further tests to define a location of the disorder. For example, the patient may be suffering from an abscess under a tooth, but may exhibit symptoms of earache. In other cases, the disorder may be multidrug resistance (MDR) secondary to treatment of a malignancy with a neoplastic agent. It is known that MDR is associated with alterations in the intracellular pH distribution.

Once the location and type of the disorder is defined, the practitioner can choose a group of drugs, known to be effective in treating the disease or disorder in the defined location, in a drug selecting step 304.

The professional may then analyze the route of uptake of the various drugs in the group. This may entail one or more of the following steps:
Mapping the delivery route from outside the patient's body to the target region in a first mapping step 306.
Mapping the target cell(s) extracellular:intracellular pH gradient and/or potential gradient in a second mapping steop 308.
Mapping the target intracellular pH distribution/gradient in a third mapping step 310.

These steps may be performed in various sequences or simultaneously.

The data from step 310 may be used to define the pH (SCP) of various organelles and sub-cellular regions in a defining step 312.

Depending on the intracellular energetic status and on the sub-cellular target of the specific disease, the optimal isoelectric point of a drug may be defined to fall in a range of values relative to the SCP.

This may be defined mathematically by:

M<|(PA drug−SCP)|<N (1).

Wherein PA drug is the pH at which the drug preferentially accumulates;

SCP is the sub-cellular pH

M and N are numeric values determined by energetic and or pH considerations from previous in vitro/in vivo experiments on the same type of cells.

Thus, in a checking step 314, the known PA value will be introduced into equation 1. The value of SCP was determined in step 312 hereinabove, and the values of M and N may be defined from previous experiments.

If the conditions of equation 1 are not met for the first drug, along a first drug delivery route, a second drug delivery route for the first drug may be checked out (not shown). Alternatively, the next step 316 is to go to another drug. This second drug may be evaluated along a second delivery route (dashed line 316-306) or alternatively, the second drug may be tested along the first delivery route (full line 316-314).

It will then be checked in step 318 to see if all the drugs in the group have been tested. If affirmative, the next step 320 is to compare the results of all the drugs tested and to choose the best drug or drugs from the group.

This may be followed by in vitro/in vivo testing (not shown).

In step 318, it may be found that not all the drugs in the group have been tested. Thus, one can proceed to the next drug in step 316.

It should be understood that various iterations may be performed to this drug testing procedure and variations of this method are deemed to be within the scope of the invention.

This type of testing procedure can be used to test a large number of drugs from the group along many different delivery routes, by repeating steps 306-314, 314-316, and or 306-318.

Additionally, the conformation of the drug, such as a protein, may be altered by chemical treatment so as to change its PA, and/or its isoelectric point. The drug may be genetically engineered to provide a different conformation. Additionally or alternatively, the drug may be formulated as a prodrug. After one or more of such manipulations, the resultant drug may be tested per steps 306-318 hereinabove.

The teachings of all the references cited in the present specification are incorporated in their entirety by reference.

It will be understood by one skilled in the art that aspects of the present invention described hereinabove can be embodied in a computer running software, and that the software can be supplied and stored in tangible media, e.g., hard disks, floppy disks or compact disks, or in intangible media, e.g., in an electronic memory, or on a network such as the Internet.

### EXAMPLES

### Example 1

A crosslinked conjugate of lactoglobulin and concavalin A was prepared in a reaction mixture containing 50 micrograms of each protein (Sigma) in 10 mM HEPES buffer (pH˜7) using 10 microliters of a 1% solution of glutaraldehyde (reaction conditions: 40° C., 10 min).

The reaction was terminated by addition of 10 microliters of 1M Tris-HCl (pH˜8)

A commercial pH gradient strip (Immmobilized pH Gradient (IPG); Amersham Pharmacia-GE) of pH range 2-10 was soaked overnight in the reaction solution and stained with Commassie Blue staining solution.

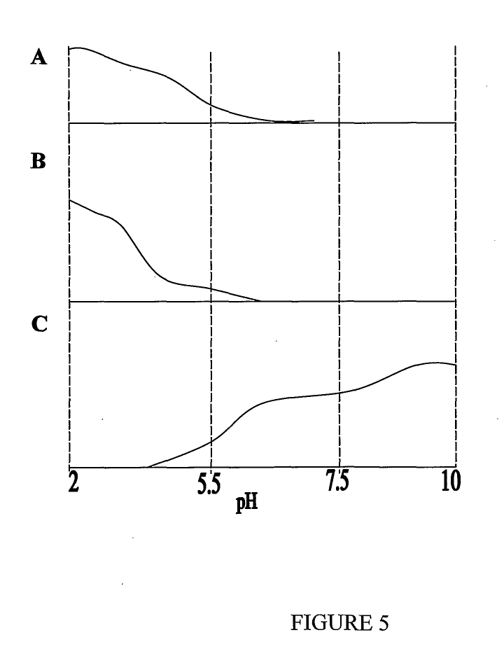

FIG. 5 shows scans representing the distribution of the individual unconjugated proteins lactoglobulin (panel A) and concavalin A (panel B), and the conjugated protein (panel C) along the 2-10 pH gradient gel.

The distribution of the conjugate protein along the pH gradient is dramatically different from that of the individual unconjugated proteins. Notably, the scan in panel C shows a strong enhancement of the protein accumulation in the pH range 5.5-7.5. i.e. within the intracellular pH range.

### Example 2

A crosslinked conjugate of lactoglobulin and bovine serum albumin (BSA) was prepared and analyzed as in Example 1.

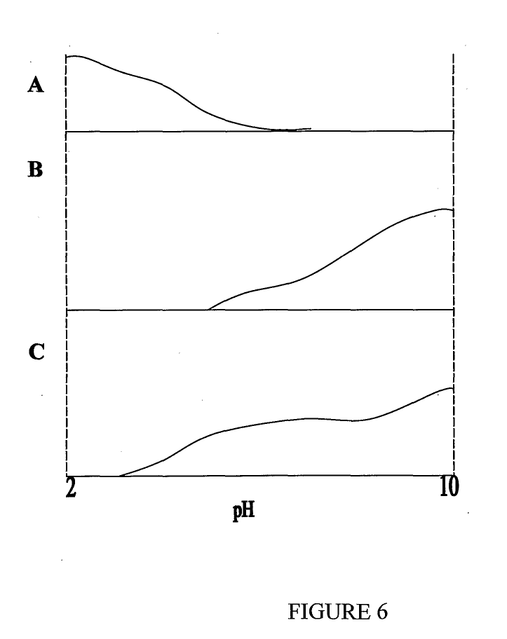

FIG. 6 shows scans representing the distribution of the individual unconjugated proteins lactoglobulin (panel A) and BSA (panel B), and the conjugated protein (panel C) along the 2-10 pH gradient gel.

The distribution of the conjugate protein along the pH gradient is significanly different from that of the individual unconjugated proteins.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1.-46. (canceled)

47. A method for targeting a drug to an intracellular location in a eucaryotic cell where the drug takes effect, which method comprises providing to a eucaryotic cell a drug and at least one moiety which shifts the trapping probability of the drug along an intracellular pH gradient, thereby causing the drug to migrate along an intracellular pH gradient to the intracellular location, wherein the drug preferentially accumulates at a pH range of the intracellular location, and wherein the drug is active or activated in the intracellular location.

48. The method according to claim 47, wherein the eucaryotic cell is a mammalian cell selected from the group consisting of a brain cell, a skin cell, a lung cell, a nerve cell, a heart cell, an alimentary canal cell, a cancer cell, a blood cell, a urinary tract cell, an infected cell, and a combination thereof.

49. The method according to claim 48, wherein the cancer cell is selected from the group consisting of a tumor cell, a leukemia cell a carcinoma cell, a lymphoma cell, a sarcoma cell, a metastatic cell, and a multidrug resistant cancer cell; or wherein the infected cell is selected from the group consisting of a parasite-infected cell, a virus-infected cell and a prion-infected cell.

50. The method according to claim 47, wherein the drug comprises the at least one moiety which shifts the trapping probability of the drug along an intracellular pH gradient; or wherein at least one drug delivery component comprises the at least one moiety which shifts the trapping probability of the drug along an intracellular pH gradient.

51. The method according to claim 50, which further comprises formulating the drug in a formulation comprising the at least one drug delivery component selected from the group consisting of a liposome, a nucleic acid vector, a sialyl Lewis receptor, folate EGF, an anti-target antibody, a pH-sensitive delivery system, a pH controlled drug release system, a time-controlled drug release system, a pressure-controlled drug release system, a molecular positive charging system, a receptor binding component, a chimeric peptide, a cathepsin-sensitive component; a buffering system, an encapsulation system, a blood-brain barrier traversing component, a component susceptible to phagocytosis, a component susceptible to pinocytosis, a component susceptible to transcytosis and a component susceptible to endocytosis.

52. The method according to claim 47, wherein the drug comprises at least one of a peptide, a protein, an enzyme, an antibody, an anti-inflammatory drug, an anti-cancer drug, an antibiotic, a drug delivery component, a sense nucleic acid, an anti-sense nucleic acid, a covalently bound adjunct, a receptor binding component, a prodrug, a cleavable sequence, or an active fragment thereof.

53. The method according to claim 47, which further comprises delivering the drug and the moiety which shifts the trapping probability of the drug along an intracellular pH gradient into a specific target tissue or cell type.

54. The method according to claim 47, wherein the pH range is less than 2 to 3 pH values.

55. The method according to claim 47, wherein the intracellular location is within a location selected from the group consisting of the cytoplasm, the cytosol, and an organelle, wherein the organelle is selected from the group consisting of a nucleus, a mitochondrion, a ribosome, a Golgi apparatus, an endoplasmic reticulum and a centrasome.

56. The method according to claim 47, which further comprises causing the drug to migrate to the intracellular location in less than 5 minutes.

57. The method according to claim 47, wherein the at least one moiety which shifts the trapping probability of the drug along an intracellular pH gradient is selected from the group consisting of a peptide, a protein and a protein fragment.

58. The method according to claim 57, which further comprises preparing a covalent conjugate of the drug and the moiety which shifts the trapping probability of the drug along an intracellular pH gradient, wherein preparing a covalent conjugate comprises at least one of: use of a cross-linking reagent; chemically conjugating the drug and the moiety, and recombinantly expressing a fusion protein comprising the drug and the moiety.

59. The method according to claim 47, which further comprises modifying the pH gradient in the cell by adding a pH modifying agent, wherein the pH modifying agent is added to the cell prior to, concurrent with or following the step of providing the drug.

60. A composition for targeting a drug to an intracellular location in a eucaryotic cell where the drug takes effect, comprising:
a drug adapted to migrate along a pH gradient to an intracellular location by having an enhanced trapping probability matched to a pH range of the intracellular location, whereby the drug is active or activated in the intracellular location;
at least one moiety which shifts the trapping probability of the drug along an intracellular pH gradient, and
an aqueous carrier.

61. The composition according to claim 60, wherein the at least one moiety which shifts the trapping probability of the drug along an intracellular pH gradient is selected from the group consisting of a peptide, a protein and a protein fragment.

62. The composition according to claim 61, wherein the drug and the moiety which shifts the trapping probability of the drug along the intracellular pH gradient are present together in a covalent conjugate; or wherein the composition further comprises at least one drug delivery component which comprises the at least one moiety which shifts the trapping probability of the drug along an intracellular pH gradient, wherein the drug delivery component is selected from the group consisting of a liposome, a nucleic acid vector, a sialyl Lewis receptor, folate EGF, an anti-target antibody, a pH-sensitive delivery system, a pH controlled drug release system, a time-controlled drug release system, a pressure-controlled drug release system, a receptor binding component, a chimeric peptide, a cathepsin-sensitive component; a buffering system, an encapsulation system, a blood-brain barrier traversing component, a component susceptible to phagocytosis, a component susceptible to pinocytosis, a component susceptible to transcytosis, a component susceptible to endocytosis and a combination thereof.

63. The composition according to claim 63, wherein the covalent conjugate is selected from the group consisting of a chemical conjugate and a fusion protein.

64. The composition according to claim 61, wherein the drug is selected from the group consisting of a peptide, a protein, an enzyme, an antibody, an anti-inflammatory drug, an anti-cancer drug, an antibiotic, a drug delivery component, a sense nucleic acid, an anti-sense nucleic acid, a covalently bound adjunct, a receptor binding component, a prodrug, a cleavable sequence, an active fragment thereof and combinations thereof.

65. A method of drug screening for a drug active at a target sub-cellular location in a mammalian cell, which comprises:
mapping an intracellular pH distribution of a cell so as to define a pH range of the target sub-cellular location;
screening drugs from a drug library to find one or more drugs having an increased probability to accumulate at a certain pH range, wherein the certain pH range is matched to the pH range of the sub-cellular location, and optionally,
testing the one or more drugs to verify an activity thereof in the sub-cellular location.

66. The method according to claim 65, wherein the screening comprises:
evaluating and sorting drugs in the drug library according to increased probability to accumulate at a certain pH range, wherein the certain pH range to form unified accumulation pH drug groups; and
matching the unified accumulation pH drug groups to the target pH range to select one or more matched drug groups.

67. The method according to claim 65, which further comprises designing a delivery system for at least one drug from the one or more matched groups suitable for delivery for the at least one drug to the sub-cellular location so as to provide at least one drug active at the sub-cellular location in the mammalian cell.
